(19) United States
(12) Patent Application Publication
(10) Pub. No.: US 2017/0209486 A1
(43) Pub. Date: Jul. 27, 2017

(54) Health-care preparation simultaneously with magnesium supplement and antioxidant functions and preparation method thereof

(71) Applicant: SHANGHAI JIAO TONG UNIVERSITY, Shanghai (CN)

(72) Inventors: Guangyin Yuan, Shanghai (CN); Jia Pei, Shanghai (CN); Wenjiang Ding, Shanghai (CN)

(73) Assignee: SHANGHAI JIAO TONG UNIVERSITY

(21) Appl. No.: 15/480,713

(22) Filed: Apr. 6, 2017

(51) Int. Cl. A61K 33/06; A61K 9/48; A23L 33/16; A61K 9/20

(52) U.S. Cl. CPC A61K 33/06; A61K 9/20; A61K 9/48; A61K 9/2095; A61K 9/4833; A23L 33/16; A23V 2002/00

(57) ABSTRACT

A health-care preparation simultaneously with magnesium supplement and antioxidant functions and a preparation method thereof are disclosed. The health-care preparation comprises a main active ingredient of magnesium agent and an excipient. The magnesium agent is MgH₂ micro-nano powder, and the excipient is a commonly used dietary supplement auxiliary material. Compared with the existing magnesium-supplementing preparation, the health-care preparation can simultaneously supplement magnesium which is the essential trace element in human body, and hydrogen which can neutralize oxygen free radicals. The present invention has the following advantages: it is convenient for storage and taking, and has a high conversion and absorption efficiency, a low cost, and no toxic side effects. Long-term use of the preparation can effectively prevent a variety of adverse symptoms caused by magnesium deficiency, moreover the introduction of hydrogen which has an antioxidant effect can benefit to anti-inflammatory, body health, anti-aging and prevention of disease.

### BACKGROUND OF THE PRESENT INVENTION

1. Field of Invention

The present invention relates to the field of dietary supplement, and more particularly discloses a health-care preparation simultaneously with magnesium supplement and antioxidant functions.

2. Description of Related Arts

With improving of the living standards, aging of the population and emerging of new life styles, the issue of health care has aroused increasing attention. It is well known that trace elements play an important role in human's health. For instance, magnesium participates in protein synthesis relevant to energy metabolism, activates and catalyzes many enzymes, regulates the nerve system, maintains the heart muscle to contract, enhances the immune function, and protects bone health. Deficiency of magnesium is seriously harmful to human health, which may cause cardiovascular disease, hypertension, diabetes, osteoporosis, migraine, anxiety disorder, depression, and so on. According to the recommended dietary allowance by Chinese Nutrition Society, an adult should take 350 mg/day of Mg for males, 300 mg/day for females, and about 450 mg/day for pregnant and lactating women. However, the daily food can hardly provide enough magnesium, and extra magnesium supplement is therefore necessary. The current magnesium nutritional supplements mainly include magnesium oxide, magnesium gluconate, magnesium citrate, magnesium salicylate, magnesium hydroxide, magnesium chloride, magnesium sulfate and other magnesium salts. However, they usually have drawbacks of low magnesium content and low efficiency of absorption (50-70%), and may cause gastrointestinal discomfort, diarrhea, chest tightness, bloody stools, mouth, face and tongue swelling, abdominal cramps and so on. Furthermore, the use of magnesium salt injection could cause injection site pain at injection site, dizziness, muscle paralysis and other adverse reactions. Therefore, it is necessary to develop new magnesium supplements with no toxic or side effects, while at the same time have higher magnesium content and higher efficiency of absorption.

In addition, studies have shown that the health state is also closely related to the oxygen free radicals in the human bodies. Excess oxygen free radicals could lead to cell damage or necrosis, and finally result in diseases and aging. Hydrogen is a colorless, odorless and non-toxic gas, which has been reported to own selective antioxidant functions by a large number of studies. It can neutralize hydroxyl radical and nitrite anion, which are two major sources causing oxidative damage. While the endogenous-specific clearance pathways have not yet been identified in human body, hydrogen can combine with reactive oxygen species to generate water, and subsequently be excreted out of human body with no toxic or other side effects. In addition, hydrogen gas shows strong penetrability, and can easily diffuse into nucleus, mitochondria and other parts of cells.

These important characteristics enable hydrogen gas to be a potential candidate to treat certain diseases. Hydrogen can protect from a variety of neurological damage, and has therapeutic effect on organ damages and diseases relevant to cardiovascular and digestive system, and also on other diseases such as diabetes, tumor radiation therapy, systemic red lupus skin lesions, rheumatoid joints inflammation and so on. Therefore, appropriate hydrogen supplementing can be of benefit to the health. In recent years, Hydrogen-rich Water containing hydrogen gas has attracted extensive attention in the international market. However, the effect of Hydrogen-rich Water is greatly limited by the low solubility of hydrogen, commonly with a general hydrogen concentration of only 0.1-1 ppm and about 5 ppm at the maximum. Low hydrogen concentration and poor stability remain the major problems that impair the therapeutic effect of hydrogen supplement products.

Currently in China there have not been found the health-care preparations or dietary supplement products for simultaneous supplement of magnesium and hydrogen, except for the hydrogen-rich water stick produced by Dr. Hayashi from Japan, whose active ingredient is based on magnesium metal. However, this product has several drawbacks: 1) It can react with water and subsequently convert to insoluble magnesium hydroxide, leading to a lower efficiency of absorption; 2) Hydrogen gas is produced as soon as it contacts with water, which is then easy to diffuse out. This makes it unstable, have to be stored carefully, and it is supposed to be taken immediately, yet the concentration of hydrogen dissolved in water is still very low; 3) Magnesium can easily convert into insoluble products, preventing further reaction with water, thus the effect would decrease significantly with the use of time. The patent CN1840497A “Hydrogen-rich water generation method and hydrogen-rich water generator” and CN201785234U “Hydrogen water generator” refer to the same active ingredient of magnesium metal, thus have the same abovementioned disadvantages. The patent CN102557227B “Calcium magnesium rich hydrogen water supplement and its preparation method” reveals a similar working principle as the hydrogen-rich water stick, using magnesium or calcium-magnesium alloy as the hydrogen-producing agent. The only difference is that an additional pH adjuster is added to promote the dissolution of magnesium, while the above-mentioned issue 2) remains unresolved.

### SUMMARY OF THE PRESENT INVENTION

Aiming to approach the above problems, the present invention provides a health-care preparation with magnesium supplement and antioxidant functions simultaneously. It uses magnesium hydride (MgH₂) micro-nano particles as the active ingredient (source of magnesium and hydrogen), which can react with water in human body and subsequently convert to magnesium ions and hydrogen gas. This invention provides a new approach for magnesium absorption and hydrogen intake. Long-term administration of this preparation can prevent from magnesium deficiency symptoms, and also can be beneficial to anti-aging and diseases prevention.

The objective of the present invention is achieved by the following technical solutions:

The present invention provides a health-care preparation with magnesium supplement and antioxidant functions subsequently, which comprises a main active ingredient of magnesium agent and an excipient. The magnesium agent is MgH₂ micro-nano powder, and the excipient is a common dietary supplement preparation auxiliary material.

Preferably, the MgH₂ micro-nano powder has a size of 100 nm to 100 μm. MgH₂ presents as micro-nano powder. For magnesium power of 100 nm-100 μm, its oxidation activity is so high that it is prone to oxidative exothermic reaction in the air, and even leads to explosion and other dangerous accidents. While after combination with hydrogen into MgH₂ micro-nano powder, it becomes very stable and safe. In the meantime, MgH₂ is also a stable and reliable source of hydrogen. As soon as MgH₂ powder enters the body through the gastrointestinal tract, it can react with water (MgH₂+H₂O→Mg²⁺+2OH⁻+2H₂) and produce Mg²⁺ and hydrogen, thereby playing a very positive role in human health.

The size range of 100 nm-100 μm is selected because it has the most economical preparation process, and is also easy to be absorbed by human body. If the particle size is less than 100 nm, the cost of preparation would increase substantially, while for the particle size exceeds 100 μm, the absorption efficacy would be undermined.

Preferably, the health-care preparation has a magnesium content of 40-400 mg/dose.

Preferably, the health-care preparation produces a hydrogen content of 72-720 mL/dose upon administration.

Preferably, the health-care preparation is in a form of tablets, pills or capsules.

Compared with the prior art, the present invention has the following advantages:

1. The health-care preparation can provide simultaneous supplement of magnesium which is the essential trace element in human body, and hydrogen which can neutralize oxygen free radicals.

2. The reaction of the prior art is as follows:

Mg+2H₂O→Mg(OH)₂+H₂

And the reaction of the present invention is further modified as follows:

MgH₂+2H₂O→Mg²⁺+2OH⁻+2H₂

As shown in the reaction formula, the hydrogen production efficiency of MgH₂ is 2 times higher than that of the metal magnesium. Moreover, MgH₂ could be directly converted into a soluble magnesium salt in human body after administration, rather than the insoluble magnesium hydroxide in the prior art. Therefore, the conversion and absorption efficiency can be greatly improved.

3. Unlike hydrogen-rich water which is limited by the low solubility of hydrogen, the hydrogen in the present health-care preparation is produced after administration, thus the hydrogen concentration is greatly enhanced, leading to a better anti-oxidation therapeutic effect.

4. The reaction products of the health-care preparation are safe and reliable, which show no toxic side effects or adverse reactions; Furthermore, the preparation is easy to store and take, and thus greatly improves the user convenience and experience.

5. The health-care preparation uses MgH₂ micro/nano-scale powder as the main active ingredient, which has a relatively low cost, and is applicable to mass production.

### DETAILED DESCRIPTION OF THE PREFERRED EMBODIMENT

The present invention will now be described in details with specific examples. The following examples can help the skilled people in this art to further understand the present invention, but not in any way limiting the present invention. It should be noted that several modifications and improvements will occur to those skilled in this art without departing from the spirit of the present invention. These are included in the scope of the present invention.

### EXAMPLE 1

The magnesium hydride is prepared as fine powder with a size of 100-200 nm. Then 250 g of the powder is mixed with 500 g of the pregelatinized starch and 250 g of the microcrystalline cellulose, and subsequently the uniform mixture is compressed into 1000 tablets of the preparation. Finally, each tablet has a weight of 1 g. 2 tablets per day should be taken for adults in 1-2 administration, and 1 tablet per day for children in 1 administration. After 1-2 weeks of continuous use, the muscle soreness in legs caused by mountaineering or long-distance running will be effectively alleviated or disappear due to the magnesium supplement.

### EXAMPLE 2

The magnesium hydride is prepared as fine powder of 400-600 mesh. Then 150 g of the powder is mixed with 150 g of mannitol, 120 g of powdered silica gel and 80 g of alginic acid, and the mixture is ground and sifted, and evenly packed into 1000 capsules. Finally, each capsule has a weight of 0.5 g. 2-3 capsules per day should be taken for adults in 1-3 administration, and 1-2 capsules per day for children in 1-2 administration. Animal experiments showed effective treatment for mice pneumonia with continuous use of the capsule powder for 2 weeks, by mixing the powder into the mouse meal, 1 capsule per day, and the pneumonia has completely healed after 3 weeks, indicating that hydrogen has effective anti-inflammatory effect by dissolving into the body fluid.

### EXAMPLE 3

The magnesium hydride is prepared as fine powder of 500 nm to 1 μm. Then 200 g of the powder is mixed with 150 g of lactose, 100 g of dextrin and 50 g of CMC, and the uniform mixture is compressed into 1000 pills of the preparation. Finally, each pill has a weight of 0.5 g. 1-2 pills per day should be taken for adults in 1-2 administration, and also 1-2 tablets per day for children in 1-2 administration. Animal experiments with guinea pigs showed effective treatment for hepatitis with continuous use of the pill powder for 1 month, by mixing the powder into the meal, 1 pill per day. The hepatitis showed obvious relief after 10 days, and completely healed after 4 weeks, indicating that hydrogen has effective anti-inflammatory and anti-antioxidant effect by dissolving into the body fluid.

### EXAMPLE 4

The magnesium hydride is prepared as fine powder of 1-10 μm. 350 g of powder is uniformly mixed with 300 g of mannitol and 150 g of microcrystalline cellulose, and compressed into tablets. And surface coated with sugar, a total of 1,000 pieces of sugar-coated tablets are obtained. Finally, each piece has a weight of 0.8 g, and 1 piece per day should be taken for adults. After 2 weeks of continuous use, the insomnia caused by neurological dysfunction will be effectively alleviated or disappear, due to the effective supplement of magnesium.

### EXAMPLE 5

The magnesium powder is prepared as powder of 1000-1500 mesh. 40 g of powder is added into 100 commercial folic acid tablets, grounded and re-compressed into tablets to obtain 100 tablets. Pregnant women should have 1 tablet per day, which will help pregnant women to supplement folic acid as well as the necessary element of magnesium. After continuous taking for 1-2 months, some gastrointestinal dysfunction symptoms due to neurological dysfunction for the elder such as abdominal fullness, bowel, sigh, eat less, vomiting, diarrhea or constipation will be effectively alleviated or disappear, due to the regular supplement of magnesium and the antioxidant effect of hydrogen.

### EXAMPLE 6

The magnesium powder is prepared as fine powder of 1000-1500 mesh. 30 g of powder is added into 100 pieces of commercial calcium supplements as the trace element additive, grounded and re-compressed into 100 tablets. 1 tablet per day should be taken for adults. Simultaneously magnesium supplementation can promote the absorption of calcium for patients with osteoporosis. After continuous taking 1-3 months, the symptoms caused by calcium and magnesium deficiency such as fatigue, cramps, backache, allergies or colds will be effectively alleviated or disappear, due to the adequate supplement of magnesium/calcium, and the anti-inflammatory and antioxidant effect of hydrogen.

The above-listed are the specific examples of the present invention. It should be noted that those skilled in this field can easily make many modifications and variations based on the present invention without any creative work. Accordingly, all the technical solutions those are based on the present invention and can be obtained by logical analysis, reasoning or limited experimentation should be included in the present invention.

What is claimed is:

1. A health-care preparation simultaneously with magnesium supplement and antioxidant functions, which comprises a main active ingredient of magnesium agent and an excipient; the magnesium agent is MgH₂ micro-nano powder, and the excipient is the commonly used dietary supplement auxiliary materials.

2. The health-care preparation with the magnesium supplement and antioxidant functions, as mentioned in claim 1, wherein the MgH₂ micro-nano powder has a size of 100 nm to 100 μm.

3. The health-care preparation with the magnesium supplement and antioxidant functions, as mentioned in claim 1, wherein a content of a magnesium element in the health-care preparation is 40-400 mg/dose.

4. The health-care preparation with the magnesium supplement and antioxidant functions, as mentioned in claim 1, wherein a hydrogen-generating content of the health-care preparation is 72-720 mL/dose.

5. The health-care preparation with the magnesium supplement and antioxidant functions, as mentioned in claim 1, wherein the health-care preparation is in a form of tablets, pills or capsules.

6. A preparation method for a health-care preparation with magnesium supplement and antioxidant functions as mentioned in claim 1, 2, 3, 4 or 5, comprising steps of: uniformly mixing MgH₂ micro-nano powder with common supplement or medicine excipients; processing with weighing, sub-material, compaction, sterilization and packaging for obtaining the health-care preparation.
